(19) United States
(12) Patent Application Publication
(10) Pub. No.: US 2021/0236978 A1
(43) Pub. Date: Aug. 5, 2021

(54) AIR PURIFICATION DEVICE

(71) Applicant: AirBiogenics, LLC, Fort Wayne, IN (US)

(72) Inventors: Miles Smith, Goshen, IN (US); Tyrone Smith, Fort Wayne, IN (US)

(21) Appl. No.: 17/222,764

(22) Filed: Apr. 5, 2021

Related U.S. Application Data
(60) Provisional application No. 63/006,231, filed on Apr. 7, 2020.

(51) Int. Cl. B01D 46/00; B01D 46/12; B01D 46/42; A61L 9/22

(52) U.S. Cl. CPC B01D 46/0028; B01D 46/12; B01D 46/0049; B01D 46/4236; A61L 2209/16; B01D 2273/30; B01D 2279/50; B01D 2279/65; A61L 2209/14; A61L 9/22

(57) ABSTRACT

An air purification device for purifying and recirculating air includes a housing, a filter mounted within the housing adjacent an inlet of the housing, a blower for drawing room air into the housing through the inlet, and an ionizer mounted at or adjacent both an exhaust portion of the blower and an outlet in the housing. Filtered air drawn into the housing by the blower is ionized immediately prior to being ejected from the housing. A silencer duct may be provided at the exhaust portion of the blower for mounting the ionizer and reducing noise.

### CROSS-REFERENCE TO RELATED APPLICATIONS

This application claims priority to U.S. Provisional Patent Application No. 63/006,231, filed Apr. 7, 2020 and titled AIR PURIFICATION UNIT, the entire disclosure of which is hereby incorporated by reference herein in its entirety.

### BACKGROUND OF THE INVENTION

### 1. Field of the Invention

The present invention relates to air purification devices and systems, and more particularly, to air purification devices and systems in which ionized particles are utilized for cleaning, disinfecting, and purifying air in an occupied space.

### 2. Description of the Related Art

Commercial air purifiers known in the art utilize various forms of air ionization technology to electrically charge air molecules and create ionized particles with positive and negative charges. The ionized particles react with and remove contaminants from the air, and can inactivate many viruses. One more recent form of air ionization technology is needlepoint bipolar ionization (NPBI). NPBI works by supplying electricity via electrodes that react with water vapor and oxygen in the air. The reactions create free radicals which are not harmful to humans, but which can capture/kill microorganisms and volatile organic compounds (VOCs), break down odors, and improve indoor air quality. An additional benefit of NPBI technology is that it does not generate harmful levels of ozone (O₃), a lung irritant which other forms of ionization technology may generate at unacceptable levels, rendering them unsuitable for occupied spaces.

While the free radicals or ions generated by NPBI technology are very useful for purifying air, they are typically only active for about 60 seconds. Thus, the distance over which such particles can be utilized to purify air is limited. When an ionizing unit providing NPBI technology is used in HVAC systems containing extensive ductwork and/or vents, the ionization treatment of air is generally more localized within the HVAC system itself, in close proximity to the ionizing unit. The NPBI ionizing unit is mounted inside the HVAC system and used to treat air drawn from a wide range of locations. The treated air is then routed to outlets of the HVAC system where it is once again mixed with room air in various occupied spaces.

It will be appreciated that by the time the treated air reaches different outlets in such a system, it may be devoid of ionized particles or have very few remaining. Thus, in such systems, the concentration of ions in room air outside of the HVAC is relatively low, if not zero, and the bulk of the air purification occurs within the HVAC system. While the speed with which ionized air is moved through an HVAC system and outputted via ventilation may be varied, increased air speed can result in increased noise and annoyance to occupants of occupied spaces.

Moreover, merely increasing airflow speed cannot not itself remedy the problem discussed above since the ions are concentrated at the ionizing unit in the HVAC system, and dramatically reduce in concentration at individual discharge vents. In short, traditional HVAC systems which incorporate NPBI ionization technology cannot simply “throw” the ions far enough or quick enough to really effectively treat the air within occupied spaces outside of an HVAC system, and instead primarily rely on treatment within the HVAC system and recirculating the treated air.

Another benefit of NPBI ionization is that if ions are introduced to room air, the ions react with particulates in the room air and cause them to agglomerate into larger molecules which then fall to the ground and/or deactivate. Such larger particles are also easier to catch by a filter. However, since the concentration of ions introduced into the room air is limited for the reasons discussed above, this additional benefit of NPBI ionization is difficult to realize in practice with conventional HVAC systems.

There is thus a need in the art for improved air purification systems which provide greater flexibility during usage, allow for easy switching of treatment areas, increase the concentration of ions discharged to and circulated within occupied spaces, reduce noise, enhance air purification techniques, increase the rate of air changes per hour, allow for greater accessibility, and improve overall user satisfaction.

### OBJECTS AND SUMMARY OF THE INVENTION

This summary is not intended to identify or point to essential features or limit the scope of the subject matter claimed herein. The present invention relates to an air purification device and methodology with at least the following objectives:

To provide an integrated standalone air purification device that accommodates ionization technology such as NPBI technology, and increases the concentration and flowrate of ionized particles outputted from the device to occupied spaces to better clean, filter, disinfect, and purify air within the occupied spaces;

To increase the velocity of ionized particles outputted from the device to occupied spaces, create better mixing of ionized particles with room air within occupied spaces, and allow for increased recirculation of ionized and agglomerated particles back to an inlet of the device for further filtration and ionization treatment;

To provide a silencer duct which reduces noise, accommodates an NPBI ionizer, facilitates ionization of filtered air as it exits a blower, and guides the ionized air out of the housing of the device directly into an occupied space;

To accommodate NPBI technology in an air purifier device at or adjacent an exhaust portion of an industrial sized blower at or adjacent an outlet of the device;

To filter particulate 0.3 microns or greater with 0.95% efficiency, and enable a user to easily replace filter packs and reduce system downtime;

To facilitate at least 1-10 air changes per hour within a commercial office space or residential area; and

To provide flexibility and ease in arranging and rearranging an array of air purification devices which accommodate NPBI technology to treat different areas within an occupied space.

In accordance with one embodiment of the invention, an air purification device for purifying and recirculating air comprises a housing having an inlet configured to guide room air into the housing, and an outlet configured to guide ionized air out of the housing. A filter is mounted within the housing adjacent the inlet for filtering the room air guided into the housing. A blower mounted within the housing defines an intake portion and an exhaust portion, and is configured to draw the room air through the inlet and the filter to form filtered air within the housing. The filtered air within the housing is then drawn into the intake portion by the blower. The blower pushes the filtered air through the exhaust portion thereof toward the outlet of the housing. An ionizer mounted within the housing is configured to ionize particles within the filtered air to generate the ionized air guided out of the housing.

In certain embodiments, the ions guided out of the housing are of sufficient concentration that a portion of the ionized air is drawn back to the inlet of the housing by the blower for further filtration and treatment.

In accordance with one aspect of the invention, the ionizer is mounted at or adjacent the exhaust portion of the blower, and may be mounted downstream of the blower. In certain embodiments, the air purification device also includes a silencer duct mounted within the housing in fluid communication with the exhaust portion of the blower. The silencer duct is configured to reduce the noise of airflow within the air purification device, and to mount the ionizer. In accordance with another aspect of the invention, the silencer duct can be configured to direct the filtered air pushed through the exhaust portion of the blower through a portion of the ionizer to the outlet of the housing. In certain embodiments, the silencer duct may include at least one baffle to reduce the noise level of the airflow.

In accordance with yet another aspect of the invention, the silencer duct may include a support surface for mounting the ionizer. The support surface defines a cutout for receiving at least one brush of the ionizer such that brush extends downwardly through the cutout, and generates charged ions in the filtered air exiting the exhaust portion of the blower. One or more nozzles may be provided within the silencer duct adjacent the brushes to spray water into the filtered air being ionized by the ionizer within the silencer duct.

In certain embodiments, the at least one baffle of the silencer duct includes a pair of baffles, and the at least one brush includes a first brush operatively disposed above and horizontally between the pair of baffles. In other embodiments, the at least one baffle may include a middle baffle and two end baffles on opposite sides of the middle baffle. The at least one brush may include a first brush and a second brush. The first brush may be operatively disposed above and horizontally between the middle baffle and one of the two end baffles, and the second brush may be operatively disposed above and horizontally between the middle baffle and the other of the two end baffles.

In yet other embodiments, a plurality of wheels may be provided which support the housing and mobilize the air purification device along a floor. A controller in operative association with at least one of the plurality of wheels communicates with a remote computing device operable by a user to mobilize the air purification device. In yet other embodiments, mounting structure is provided for mounting the air purification device to a wall or ceiling.

In accordance with yet another aspect of the invention, the ionizer operates at a voltage less than or equal to 11.7 electron volts, and is ozone free technology (e.g., certified by UL 867 and UL 2998 to be ozone free). The ionizer may produce ions with an ozone concentration in the range of 0 to 0.05 ppm (50 ppb), or in the range of 0 and 0.005 parts per million (5 ppb).

In another embodiment of the invention, an air purification system is provided for purifying and recirculating air, and includes a first array of the air purification devices described above. A first of the first array of air purification devices and a second of the first array of ionization devices are arranged such that ionized air exiting the outlet of the housing of the first of the first array of purification devices is received in the inlet of the second of the first array of purification devices. In this manner, the array of purification devices of the air purification system can work in harmony to filter, ionize, purify, and recirculate air within an occupied space.

In accordance with another aspect of the invention, the air purification system may include a second array of air purification devices. The first and second array of air purification devices may be configured as standalone units spaced apart from one another, and the arrays of purification devices may be fluidly coupled by a fluid duct.

In accordance with yet another embodiment of the invention, a method of purifying and recirculating air comprises drawing room air through an inlet of a housing and a filter mounted in the housing to form filtered air within the housing, drawing the filtered air to an intake portion of a blower in the housing, pushing, by the blower, the filtered air toward an outlet of the housing, ionizing, via an ionizer, filtered air exiting an exhaust portion of the blower to form ionized air, and guiding, via an outlet in the housing, the ionized air out of the housing.

In accordance with one aspect of the invention, the ionized air contains ozone in a range of 0 to 0.05 ppm (50 ppb), or in a range of 0 to 0.005 parts per million (5 ppb). In accordance with another aspect of the invention, the blower is configured to push the ionized air (electrically induced ions) out of the housing at a flow rate in a range of 340,000,000 ions/cm3/second to 400,000,000 ions/cm³/second, or up to approximately 1.9×10¹¹ ions/Ft.³/min. In certain embodiments, the blower may push ionized air having more than 400,000,000 ions/cm³/second.

In certain embodiments, the method further includes guiding the filtered air exiting an exhaust portion of the blower through a silencer duct with the ionizer mounted to the silencer duct.

Various other objects, advantages, features, and characteristics of the present invention, as well as the methods of operation and functions of related structural elements, and the combination of parts and economies of development and manufacture, will become readily apparent to those of ordinary skill in the art upon consideration of the detailed description below with reference to the accompanying drawings, all of which form a part of this specification.

### BRIEF DESCRIPTION OF THE DRAWINGS

The drawings are not intended to limit the scope of this invention, which is set forth with particularity in the claims as appended or as subsequently amended, but merely to clarify and exemplify the invention. Accordingly, a further understanding and a more complete appreciation of the present invention and many of the attendant aspects thereof may be readily obtained as the same becomes better understood by reference to the following detailed description, when considered in conjunction with the accompanying drawings, where:

FIG. 1 is a front cutaway perspective view of an assembled air purification device in accordance the invention;

FIG. 1A is an enlarged view of the front vent and a portion of the silencer duct of the air purification device of FIG. 1, showing the brushes and water nozzles disposed between adjacent baffles;

FIG. 2 is rear cutaway perspective view of the assembled air purification system of FIG. 1;

FIG. 3 is an exploded view of the air purification system of FIG. 1, showing a silencer duct, an ionization unit, air filters, filter pack covers, and the front cover removed from the device;

FIG. 4 is a perspective view of a first support bracket for mounting the air purification system of FIG. 1 to a wall;

FIG. 5 is a perspective view of a second support bracket for mounting the air purification system of FIG. 1;

FIG. 6 is an enlarged front perspective view of the assembled silencer duct and ionization unit of the air purification system of FIG. 1 in accordance with the present invention;

FIG. 7 is an exploded front perspective view of the silencer duct of FIG. 6, showing the cutout in a support surface which supports the ionizer unit and receives the brushes of the ionizer unit;

FIG. 8 is a front perspective cutaway view of the air purification system of FIG. 1 in operation, showing ionized particles exiting the system into room air, and larger agglomerated particles forming and falling to the ground and/or reentering the device;

FIG. 9 is a side perspective view of the air purification of FIG. 1 operatively disposed in a remote controlled, power-driven cart;

FIG. 10 is a side perspective view of the air purification of FIG. 1 operatively disposed in a pushcart;

FIG. 11 is a schematic diagram of an air purification system in which two arrays of air purification devices are arranged around a perimeter of a warehouse and work in harmony to purify the air within the warehouse;

FIG. 12 is a front, perspective, cutaway view of an alternate embodiment of the air purification system of the present invention, showing a housing with inlets on opposite sides thereof, a blower mounted within the housing between the inlets with no silencer duct attached thereto, and an ionizing unit mounted directly to the side of the blower adjacent an exhaust portion of the blower;

FIG. 13 another embodiment of the present invention in which two air purification standalone devices in accordance with FIG. 1 are stacked on top of one another; and

FIG. 14 is another alternative embodiment of the present invention in which an air purification device having a silencer duct accommodates an ionizer and is operatively disposed within an air duct adjacent an end of the air duct for exhausting ionized air out of the end of the air duct and into an occupied space.

### DETAILED DESCRIPTION OF THE PRESENT INVENTION

The present disclosure is not intended to be limited to the specific terminology so selected, and it is to be understood that each specific element includes all technical equivalents which operate in a similar manner. Specific embodiments that may be practiced are shown by way of illustration and explanation. The embodiments are described in sufficient detail to enable those skilled in the art to practice the embodiments, and it is to be understood that logical, mechanical, and other changes may be made without departing from the scope of the embodiments. The following detailed description is therefore not to be taken in a limiting sense. In describing exemplary embodiments of the present invention illustrated in the drawings, specific terminology is employed for sake of clarity.

The air purification device described herein is a portable standalone device that can function in virtually any indoor space, accommodate NPBI ionization technology or other ionization technologies that create ions in air, and be used either as the sole device to purify air in an occupied space or in conjunction with current indoor air quality (IAQ) products already used, including, for example, existing HVAC systems which may or may not employ their own purification technologies.

By placing NPBI ionization technology directly at or adjacent the exhaust of a commercial blower, the air purification device is able to spot-treat any desired space and increase the flow rate and concentration of harmless ions directly injected into the occupied space for treatment of air therein while remaining UL 867 and UL 2998 compliant (e.g., having ozone concentrations which do not exceed 0.05 ppm (50 ppb), or which do not exceed 0.005 parts per million (5 ppb)). As further discussed below, MERV 1-15, D.O.P. (MERV 16) filters, carbon filters, and HEPA filters may also be utilized to filter out contaminants and reduce odors.

The commercial blower of the air purifier device facilitates not only an increased flow rate of harmless ions injected directly into an occupied space, but also an increased flow rate of room air into the device through suction, and by extension, recirculation of ionized air and agglomerated particulates back into the device for further filtration. While a typical HVAC system which runs twenty-four hours, seven days a week may treat all of the air in a commercial space over time, such treatment takes significantly longer and cannot “spot treat” a given space, particularly when other factors come into play, such as windows and doors being opened and closed, a particular pollutant or contaminant being introduced to a space occupied by people, etc.

By using a silencer duct specifically designed for placement at the exhaust portion of the blower, and mounting the ionization technology to the silencer duct directly in the path of filtered air exiting the blower, the purification device greatly improves the rate of air recirculation and ion purification treatment that can be accomplished in an occupied space. The purification device is able to change over the air in a room up to ten times per hour or more, far more than exiting ionizers, while also reducing noise levels which would otherwise be unsuitable for certain work spaces. The portability of the purification device disclosed herein allows it to be mounted to virtually any location on the floor, wall, countertop, or ceiling of a room, simply placed on the floor unconstrained, and/or moved by a pushcart or remote controlled cart within an occupied space.

The phrases “occupied space” and “occupied spaces” used herein refer to any form of indoor space or spaces, including but not limited to a commercial space, a residential space, or a storage space capable of being occupied by a person, equipment, or any form of storage materials. The purification devices, systems, and methodologies described herein improve air purification of such occupied spaces.

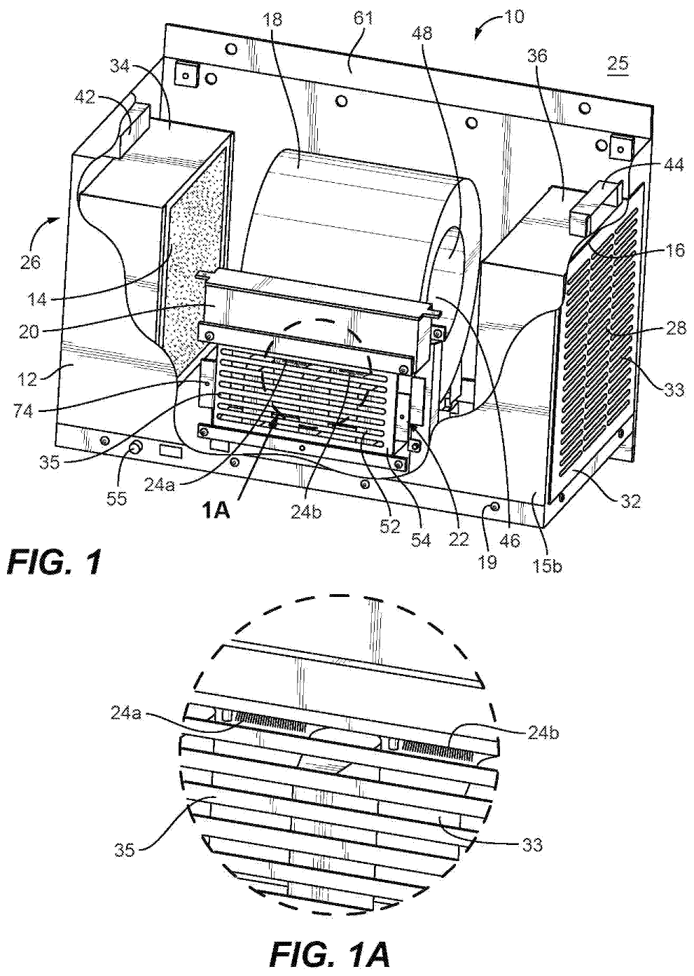

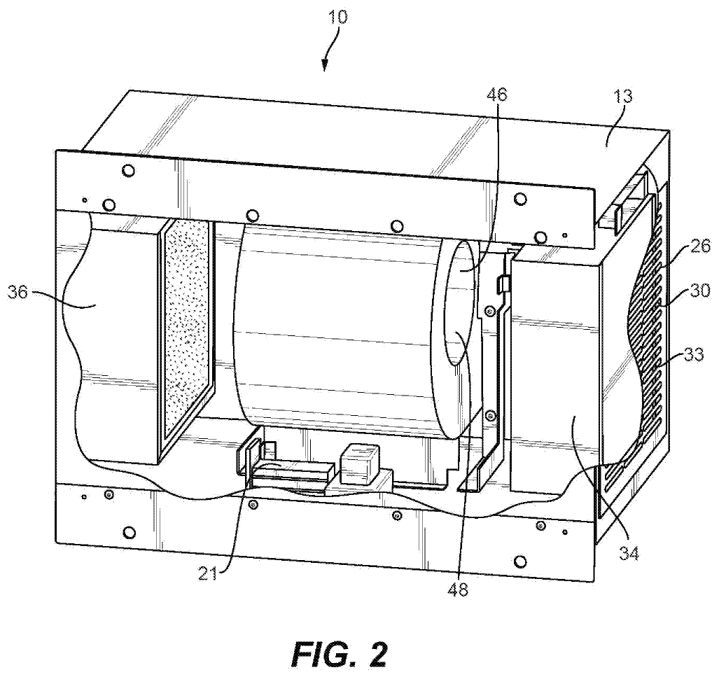

Referring to FIGS. 1-3, an air purification device 10 in accordance with the present invention includes a generally rectangular-shaped housing 12, filters 14, 16 operatively disposed within housing 12 on opposite sides thereof, a blower 18 centrally mounted within housing 12, and an ionizer 20, also mounted within housing 12. The air purification device 10 may also be equipped with a silencer duct assembly 22 downstream of blower 18. The silencer duct assembly 22 mounts ionizer 20 and positions brushes 24a, 24b of ionizer 20 within one or more flow paths of filtered air exiting blower 18 in order to ionize the filtered air 18 immediately before it exits housing 12.

Housing 12 may be formed with a top panel 13, side panels 15a, 15b, 15c, 15d, and a bottom panel 17, and is configured to support and enclose blower 18, ionizer 20, and silencer duct assembly 22. Some of panels 13, 15a, 15b, 15c, 15d, 17 may be integrally or separately formed, or connected to one another in any suitable manner whereby access to the interior of housing 12 can be achieved by user manipulation of housing 12 (e.g., by removing or pivotally moving one or more of the panels). Screws 19 and one or more support beams or brackets (not shown) may additionally be utilized to mount the panels together. Housing 12 may be formed in any suitable size and shape to store and enclose blower 18, ionizer 20, and silencer duct assembly 22. Housing 12 may also be formed from any desired material, including extruded aluminum framing, formed sheet steel, and/or plastic polymers. Handles 42, 44 may be attached to housing 12 for carrying air purification device 10.

As shown, air purification device 10 is a completely standalone device except for electrical power supplied to blower 18 and ionizer 20, optional heating and/or a/c components (not shown), and optional water supplied to nozzles to moisturize air within housing 12 (further discussed below). Air purification device 10 is configured to guide room air 25 into housing 12 through inlets 26, 28 on opposite sides thereof. Inlets 26, 28 may be provided with side plates 30, 32 which form a portion of side panels 15a, 15c, or which form the entire side panels 15a, 15c. Side plates 30, 32 define slots 33 for guiding room air 25 into housing 12. Slots 33 may be defined at an angle (e.g., a 15° angle upward or downward) depending on whether air purification device 10 is to be installed on a floor or mounted to a wall, ceiling, or other structure within an occupied space of a residential or commercial building. Inlets 26, 28 may alternatively be configured with a grill having an adjustable louve that can be reconfigured to positions between a fully open position and a fully closed position to vary the inlet velocity of room air 25 at inlets 26, 28 at a given blower 18 setting.

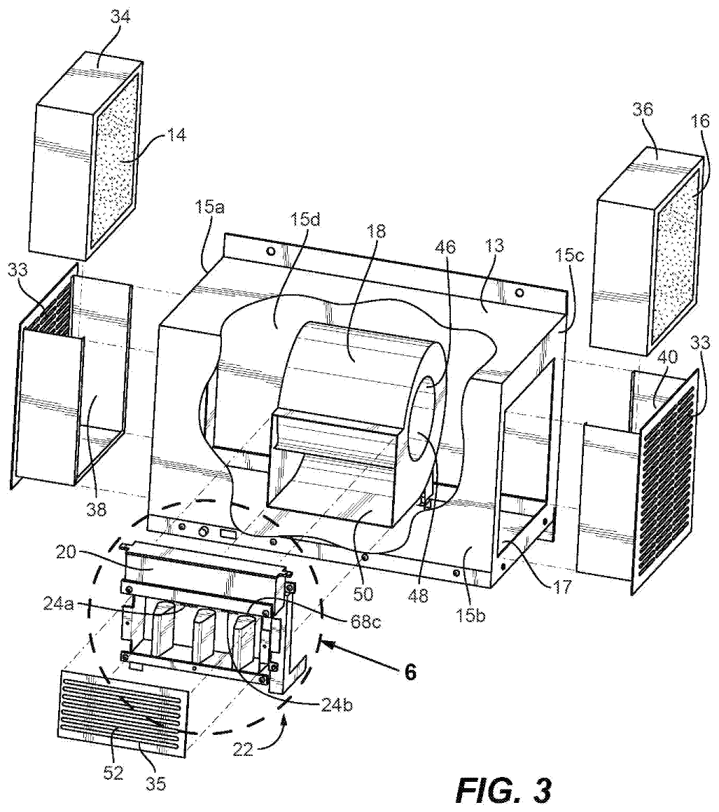

Inlets 26, 28 guide room air 25 into housing 12, through slots 33, into U-shaped filter covers 34, 36 which house filters 14, 16 in confined spaces via a press or interference fit. As shown in FIG. 3, filter covers 34, 36 are removable along with filters 14, 16 to transport used filters 14, 16 without direct contact therewith. Filter covers 34, 36 may be manufactured as reusable pieces which receive replaceable filters, or as or disposable (modular) replaceable pieces themselves.

Various types of bi-lateral and/or uni-lateral filters 14, 16 may be individually or collectively utilized, including, for example, MERV 1-15, D.O.P. (MERV 16) filters, MERV 17-MERV 20, a HEPA filter (0.5 or less microns), carbon filters, pleated pre-filters with a MERV 8 or greater filtration rating, and the like. Charcoal filters for odor removal may also be utilized. A varying number of filters 14, 16 may be placed in each filter cover 34, 36 as filter packs. As an example, one to six filters may be placed in each filter cover 34, 36 depending on the particular application or environment in which the air purification device 10 is placed, and/or the size of air purification device 10 utilized. Additional filters may be optionally placed throughout the interior of housing 12, individually or in filter packs. Such filter packs may be designed to house the air-filters for a “quick-change” system that allows for ease of maintenance. Filters 14, 16 may also be provided with a “vanity” to protect them and give them an aesthetic appearance. In certain embodiments, air purification device 10 may include a magnehelic gauge (not shown) to monitor filter life and determine any required maintenance that may be needed.

Filters 14, 16 may be placed in filter covers 34, 36 by removing filter covers 34, 36 from air purification device 10 and press-fitting filters 14, 16 into spaces 38, 40 defined by filter covers 34, 36. Filters 14, 16 are then sandwiched, encapsulated, and sealed within filter covers 34, 36 within housing 12. It will be appreciated that as blower 18 draws/pulls room air 25 through slots 33 into confined spaces 38, 40 (FIG. 3), the air is entirely forced through filters 14, 16 within U-shaped covers 34, 36, and exits on opposite sides thereof within housing 12. This filtered air is further pulled/drawn toward blower 18 at a central portion of housing 12.

Blower 18 is preferably centrally mounted within housing 12 and equipped to receive the filtered air which exits filters 14, 16 within housing 12 on opposite sides thereof. Blower 18 includes a concave, cylindrically-shaped intake portion 46 defining an internal cylindrical channel 48 horizontally through the center of blower 18. Channel 48 receives the intake filtered air and pulls it through slots defined therein (not shown) radially outward into blower 18. Blower also pushes the filtered air drawn in at intake portion 46 out through an exhaust portion 50 (FIG. 3) at a relatively high velocity toward an outlet 52 of housing 12.

Various types of blowers 18 may be utilized. Blower 18 is preferably a commercially available blower which includes inlet(s) and outlet(s), a fan, and a motor for driving the fan. Blower 18 may be, for example, an inverted 1 HP, single phase, 110V 3,000 CFM 3-speed blower motor unit, with thermal overload and a double inlet forward curve. Alternatively, a standard PSC motor or an ECM motor/blower assembly may be utilized.

Blower 18 is preferably centrally mounted within housing 12 between inlets 26, 28 adjacent outlet 52. In other embodiments, a smaller air purification device 10 can be utilized with an in-line 4″ thru 10″ blower operating on 110V. Blower 18 speed/pressure may be adjusted using a potentiometer 55 to regulate air-flow. Such smaller blowers may be rated up to 1,200 CFM, and may utilize an optional carbon pre-filter in conjunction with a MERV 8 or greater filter, along with a HEPA filter. Blower 18 can range from 500 CFM to 3,000 CFM, and can be electrically coupled via power block 21 to a power source such as, for example, a 115V, 60 HZ, single phase and 15.3 amps power source. Power block 21 may also be utilized by ionizer 20 to electrically couple to a power source. The power source may alternatively include a standard 110V/120V circuit and 220V/230V circuitry. Power block 21 may include a fuses block, end barriers, and appropriate terminal blocks, as well as insulated male/female quick-disconnects and wire ferrules for correct wiring and routing.

Once filtered air passes through blower 18, it is ejected through outlet 52 of housing 12 under pressure from blower 18. Outlet 52 may be formed from a front plate 54 similar to side plates 30, 32, and may include slots 35 for discharging the filtered air pushed by blower 18 back into the occupied space outside of housing 12 to further mix with room air 25. As described above, potentiometer 55 may be utilized for adjusting the blower pressure, and thus the velocity at which filtered air is ejected from blower 18 toward outlet 52. Additionally, similar to inlets 26, 28, outlet 52 may be configured with a grill having an adjustable louve that can be reconfigured to positions between and including a fully open position and a fully closed position to vary the outlet velocity of the filtered ionized air.

The filtered air within housing 12 is ionized immediately before being ejected through outlet 52. Various forms of ionization technology may be used with air purification device 10 in the ionizing unit 20 (also referred to herein as an ‘ionizer’), and mounted within housing 12 at or adjacent blower 18, preferably at at or adjacent exhaust portion or exhaust flange 50 of blower 18 or slightly downstream thereof. Ionizer 20 may be, for example, any desired needle-point bi-polar ionization system, modular unit, or assembly having a carbon brush electrode coupled with a flexible circuit capable of producing a maximum EV of 11.7. Ionizer 20 may be UL 867 and UL 2998 compliant (e.g., ozone concentrations which do not exceed 0.05 ppm (50 ppb), or which do not exceed 0.005 parts per million (5 ppb)). Ionizer 20 may alternatively be configured as flexible ionization bar. Ionizer 20 may utilize, for example, Global Plasma Solutions (GPS) NPBI, which is bi-polar ionization that creates Cold Plasma discharged particles in the form of protons (H+) and (O2—) ions.

Creating Cold Plasma discharged particles using ionizer 20 may filter out viruses while remaining UL 867 and UL 2998 compliant. For example, air purification device 10 may neutralize airborne COVID-19, FcoV, H1N1, H5N1, SARS, Coxsackie, and/or MRSA through various mechanisms. It will be appreciated that such mechanisms include the positive charge (H+) and negative charge (O₂—) ions surrounding a hemagglutinin (Glycoproteins surface—envelope) that forms an organism triggering infection. The ions change into highly reactive hydroxyl radicals which, when contacting the hemagglutinin (e.g., the peplomer—Glycoprotein spike), can destroy the peplomer (Glycoprotein spike) at the molecular level, potentially rendering the virus ineffective. In this manner, air purification device 10 may render many molecules ineffective at 11.7 eV or below, including harmful VOCs, such as Acetone 9.69 eV, Butanal 9.73 eV, Carbon Disulfide 10.08 eV, Dichlorobenzene 9.07 eV, Ethanol 10.62 eV, Formaldehyde 10.87, Toluene 8.82 eV, Xylene 8.56 eV, and/or Methylene Chloride 11.28 eV.

Examples of certain Global Plasma Solutions (GPS) NPBI technology and devices are described in, for example, U.S. Pat. No. 9,025,303, titled ION GENERATION DEVICE, and U.S. Pat. No. 10,695,455, titled FLEXIBLE ION GENERATOR DEVICE, which are hereby incorporated by reference herein in their entireties. Examples of ionizers that may be utilized as ionizer or ionizing unit 20 include, for example, GPS's Model # GPS-FC48-AC™—Compact Auto-Cleaning Ionization System, GPS's Model # GPS-FC24-AC™, and similar such models.

Other forms of ionization technology may be utilized in ionizer 20, including, but not limited to, UVC (ultra-violet radiation/light), PCO (photo catalytic oxidation), CDT (corona discharge tubes), and DBD (dielectric barrier discharge). These technologies may be utilized together or independently. It will also be appreciated that use of UVC technology requires the proper cubic feet per min (CFM) of airflow, which is pathogen specific, the proper Polytetrafluoroethylene (PTFE), a telflon coating for reflection in the air stream, the proper UVC dose in J/M² or mJ/M², which is also pathogen specific, and the proper latent time/exposure time for UVC to treat pathogen(s) and inactivate them. Additionally, the size of a pathogen to be treated is also a consideration when using ionic technologies such as UVC, which is pathogen dose-specific.

Ionizer 20 is configured to ionize particles with positive and negative charges within pressurized filtered air exiting exhaust portion 50 of blower 18 to form the pressurized ionized air that is forced through outlet 52. Ionizer 20 may include downwardly extending brushes 24a, 24b having bristles that provide an electrostatic charge to air particles within the filtered air exiting exhaust portion 50 of blower 18.

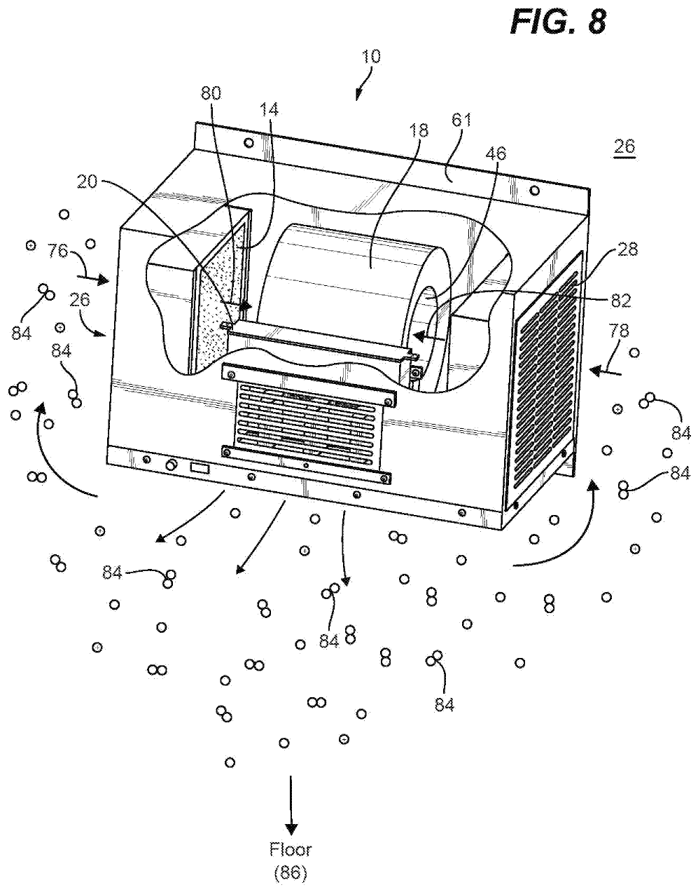

It will be appreciated that this configuration of housing 12, inlets 26, 28, filters 14, 16, blower 18, ionizer 20, and outlet 52 (where filtered air internal to housing 12 is ionized in the exhaust stream of the blower 18 immediately prior to being ejected through outlet 52), the air purification device 10 facilitates a dramatic increase in the concentration, flow rate, and recirculation of ions mixing with room air 25. As further described below with respect to FIG. 8, the increased concentration of ions enables better treatment and purification of room air 25 within an occupied space outside of purification device 10 due to the increased agglomeration and deactivation of contaminants in the room air 25 which fall to the floor, as well as recirculation of the agglomerated particles back to inlets 26, 28 for further filtration by air purification device 10.

### Silencer Duct Assembly

As discussed above, one of the issues with moving air at high velocities is the increased noise the airflow creates. As best shown in FIGS. 3 and 6-7, in certain embodiments, air purification device 10 may be provided with a silencer duct assembly 22 mounted at exhaust portion 50 of blower 18 (e.g., mounted to the exhaust flange of blower 18) or slightly downstream thereof. Silencer duct assembly 22 includes an upper horizontal mounting bracket 56 and side rails 58, 60 for mounting and supporting ionizer module 20, whereby ionizer unit 20 is also mounted at or adjacent exhaust portion 50 of blower 18. As shown in FIG. 7, upper horizontal mounting bracket 56 defines a rectangular cutout region 62 configured to receive downwardly extending brushes 24a, 24b when ionizing unit 20 is mounted to upper horizontal mounting bracket 56.

Silencer duct assembly 22 also includes a bottom horizontal mounting bracket 64 which supports three vertically extending baffles 66a, 66b, 66c arranged side by side, as well as side end brackets 67, 68 attached to or integrally formed with bottom horizontal mounting bracket 64. Baffles 66a, 66b, 66c are configured to slow filtered air exiting exhaust portion 50 of blower 18 to reduce a noise level of airflow within air purification device 10. End baffle 66a, middle baffle 66b, and end baffle 66c have convex rounded proximal ends 68a, 68b, 68c and smaller convex roundel distal ends 70a, 70b, 70c. Baffles 66a, 66b, 66c are tapered from proximal ends 68a, 68b, 68c, which have a relatively larger radius, to the smaller distal ends 70a, 70b, 70c, which have a relatively smaller radius as shown.

Baffles 66a, 66b, 66c and end brackets 67, 68 of silencer duct 22 define four channels or flow paths A, B, C, and D for airflow through silencer duct 22. It will be appreciated that the smooth rounded surfaces of convex proximal ends 68a, 68b, 68c split and slow filtered air pushed through exhaust portion 50 of blower without jarring the air or rapidly changing its direction, and guide the filtered air into the four channels A, B, C, D. Frictional interfaces of the airflows through channel A at end bracket 67 and end baffle 66a, through channel B at end baffle 66a and middle baffle 66b, through channel C at middle baffle 66b and end baffle 66c, and through channel D at end baffle 66c and end bracket 68, can produce laminar airflow through channels A, B, C, D with higher velocity airflow at respective centers of these flowpaths.

It will also be appreciated that silencer duct assembly 22 will reduce the noise level produced by the filtered air as it reaches outlet 52 of housing. However, while the noise level is reduced due to the reduced speed of the airflow and/or any induced laminar flow thereof, the noise level for a given velocity or flowrate of the air will be lower than what it would be for the same velocity or flowrate of the air without the silencer duct assembly 22. Thus, silencer duct assembly 22 allows for a higher flowrate at a given noise level (e.g., decibel output). Other silencing devices may be utilized, such as, for example, a 6-8 decibel silencer insulating material.

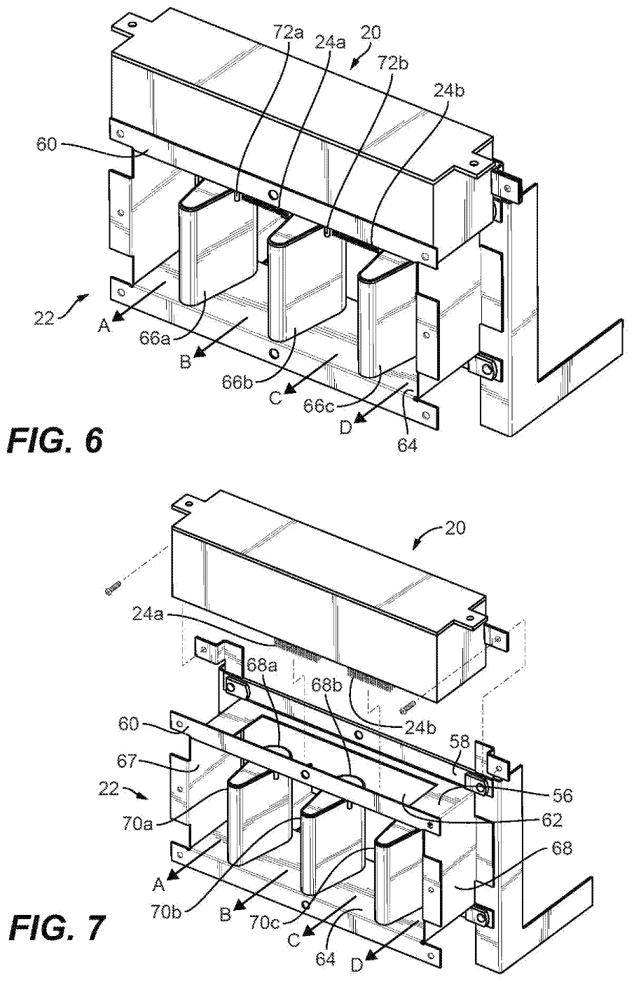

As shown in FIG. 6, when ionizer 20 is mounted to upper horizontal bracket mounting bracket 56 of silencer duct assembly 22, brushes 24a, 24b extend downwardly through cutout region 62 with brushes 24a, 24 relatively centered within channels B and C, respectively, where the velocity of air will be highest (e.g., above but horizontally between end baffle 66a and center baffle 66b and horizontally between center baffle 66b and end baffle 66c). In this manner, brushes 24a, 24b of ionizer can produce ions within high velocity air flows through silencer duct 22. These high velocity ionized air flows are then discharged through outlet 52.

It will be appreciated that brushes 24a, 24b may also ionize air flowing through channels A and D, and that a single brush or more than two brushes may be utilized. Silencer duct assembly 22 may also be modified so that brushes 24a, 24b are disposed vertically lower between adjacent baffles. Other shapes and mounting structures may be utilized. As best shown in FIGS. 6-7, silencer duct assembly 22 may also include water nozzles 72a, 72b operatively disposed adjacent brushes 24a, 24b, respectively. Water nozzles 72a, 72b are configured to spray water into the filtered airflows in channels B and C as they are ionized by ionizing unit 20. This moisturizes the filtered air as it is ionized, and may facilitate ionization and/or a humidification function. Additional nozzles and brushes may be utilized. Nozzles 72a, 72b may be user-operable by pushbutton 74 or other control on housing 12. In certain embodiments, a dehumidifier may also be provided within silencer duct 22 or housing 12 generally.

It will be appreciated that in other embodiments, air purification device 10 may be equipped with heating devices such as heating coils to heat air in housing 12, either prior to or during its discharge from blower 18 through outlet 52 of housing 12. In yet other embodiments, air conditioning capability may be provided to the various air purification devices and systems described herein. By way of example, the air purification device could be inserted into a window with the filters removed. One or more inlets or exhaust ports could be defined and selectively sealed or opened on a rear side of the housing opposite the blower, the blower could be run in reverse, and/or the device could be equipped with a separate duct to independently discharge heat to outside air regardless of whether the filters are removed. A separate internal chamber could be maintained within the housing for retaining air to be treated.

As shown in FIG. 8, during operation, blower 18 draws intake room air 25 into inlets 26, 28 at arrows 76, 78. The intake air passes through filters 14, 16 and reaches intake portion 46 of blower 18 on opposite sides thereof at arrows 80, 82. The intake air 80, 82 is then pressurized and pushed out exhaust portion 50 of blower and guided through silencer duct assembly 22 where it is ionized as described above, and expelled from housing 12 through outlet 52 at a high velocity.

When the ionized filtered airflow exits housing 12 at a high velocity and encounters additional room air 25, it reacts with particulates therein and causes agglomeration of particles 84 to occur. Many of these agglomerated particles 84 will fall to the floor 86 of the occupied space and become deactivated. Other of these agglomerated particles 84 will be recirculated back and sucked into inlets 26, 28 (due to high suction by commercial blower 18) where they will be caught by filters 14, 16. Due to the high velocity at which the ions are expelled from housing 12, they will shoot through the occupied space, bounce off of walls and ceilings of the room, and eventually be pulled with room air 25 back to inlets 14, 16 by the high suction from blower 18 or fall to the floor 86.

Thus, due to the increased ion flow rate into the room air, more particulate is deactivated outside of housing 12, more agglomerated particles are produced which fall to floor 86, and more agglomerated particles are produced which recirculate back to inlets 14, 16 for further filtration. It will also be appreciated that since a higher concentration of ionized particles will be pulled back to inlets 26, 28, particulate already caught in filters 14, 16 of air purification device 10 may be further treated by ions returning back to inlets 26, 28.

In short, when remaining particulate in room air is agglomerated, the molecular weight of the particulate is enlarged, which causes one of two outcomes: (1) the particulate is inactivated and captured by filters within the air purification device 10 upon entry thereof, or (2) the particulate is inactivated and falls to the ground. This continuous recycling of ions does not stop. As described above, since ions are only active for 60 seconds, the proper positioning and mounting of the ionization technology disclosed herein vastly improves these processes. Indeed, the air purification device 10 described herein can produce up to and including 400 million ions per cubic centimeter per second. For example, the concentration of ions produced may be between 340,000,000 to 400,000,000 ions/cm³/second. These ion concentrations can inactivate certain viruses, bacteria, mold, and VOCs.

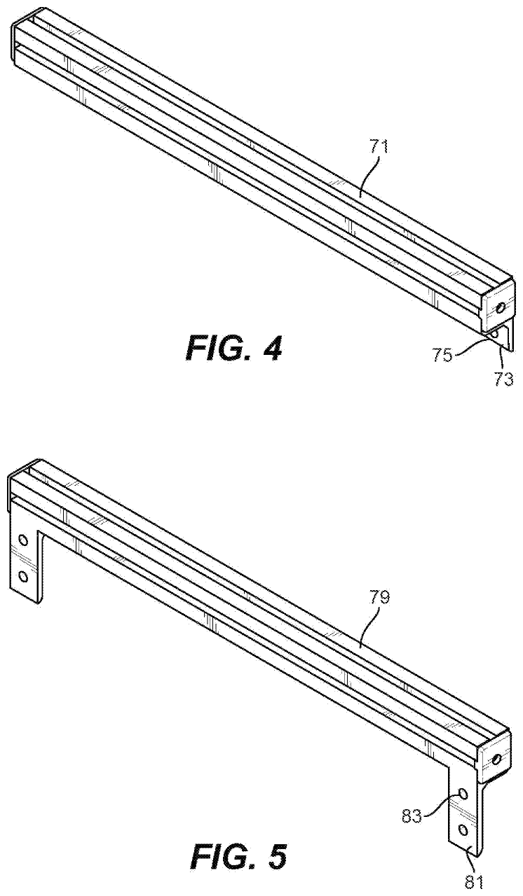

The air purification device 10 may be mounted using, for example, mounting bracket 61 integrally formed with or separately attached to housing 12. Various types of mounting brackets 71, 79 (FIGS. 4-5) having through-holes 75 and flanges 73, 81 may additionally or alternatively be used to mount air purification device 10 to a wall, countertop, ceiling, column, or other internal structure. Air purification device 10 may alternatively be simply placed on a floor or in an elevated position atop any suitable support structure or countertop.

### Other Configurations/Arrangements of Air Purification Device

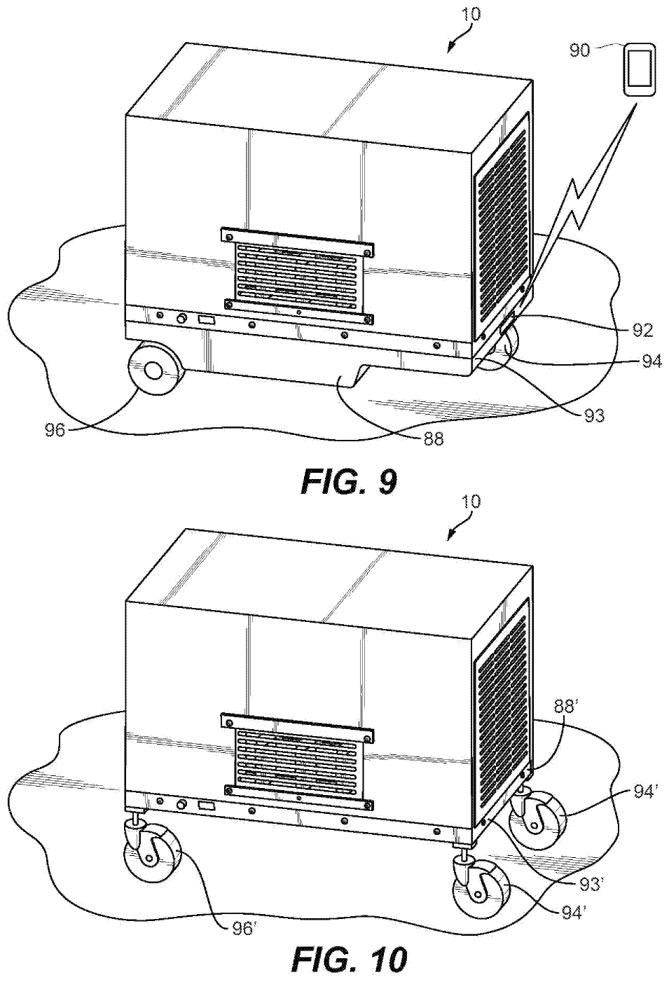

Other configurations and arrangements of the air purification device described above may be utilized. By way of example, as shown in FIG. 9, in one embodiment, air purification device 10 may be placed on a remote-controlled motorized dolly 88 having a front wheel 93 and a pair of rear wheels 96. Front wheel 94 may be motor driven and controllable by a remote computing device 90 in operative communication with controls 92 on dolly 88. In this manner, air purification device may be mobilized through a contaminated area by remote control to purify the contaminated area. As air purification device 10 is a standalone portable unit, in certain embodiments, both the blower and the ionizer may be battery powered. In other embodiments, a power source may supply power to power block 21, and air purification device 10 may be remotely moved over small distances to spot treat an area. In yet other embodiments, dolly 88 may be self-driving, whereby air purification device 10 automatically cleans an area. In yet other embodiments, air purification device 10 may be placed on a pushcart 88′ (FIG. 10) having front wheels 94′ and rear wheels 96′, and manually pushed between and around different locations or occupied spaces. In other embodiments, air purification device 10 may be communicatively linked to a central control system, cabinet, or safety protocol system, and centrally controlled at one location.

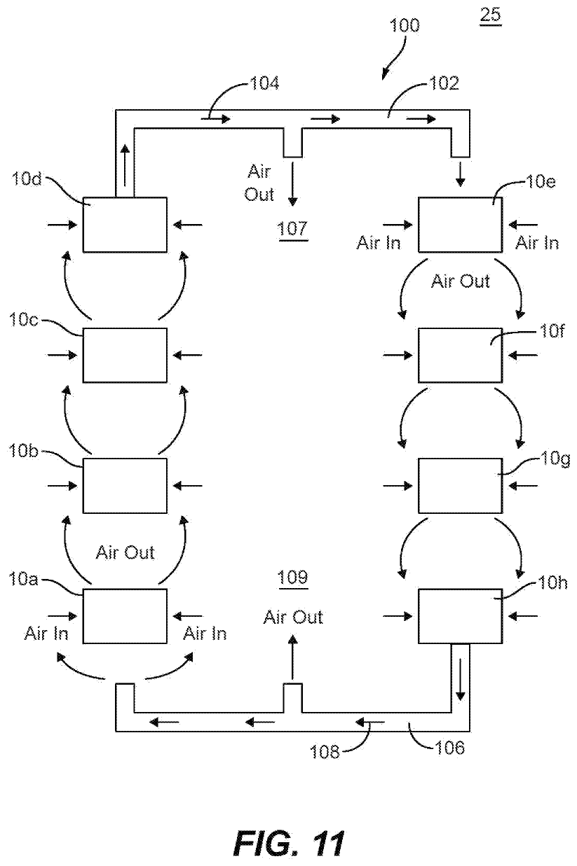

Referring to FIG. 11, an air purification system 100 in accordance with the invention includes a first array of air purification devices 10a, 10b, 10c, 10d and a second array of air purification devices 10e, 10f, 10g, 10h identical or similar to air purification device 10, and operatively disposed along two sides of a perimeter or other area of an occupied space such as a warehouse. As shown, the first and second arrays of air purification devices 10a-10d, 10e-10h are separated from one another, but arranged in series so that some of the filtered ionized air exiting one device mixes with room air 25 and enters the inlets of the next adjacent device in the array. The two arrays may be partially fluidly coupled by fluid ducts 102, 106 attached to respective outlets of air purification device 10d and air purification device 10h as shown. Fluid ducts 102, 106 may also be configured to deposit treated ionized air in more central locations 107, 109 for recirculation in the occupied space. The devices may be moved and rearranged as needed.

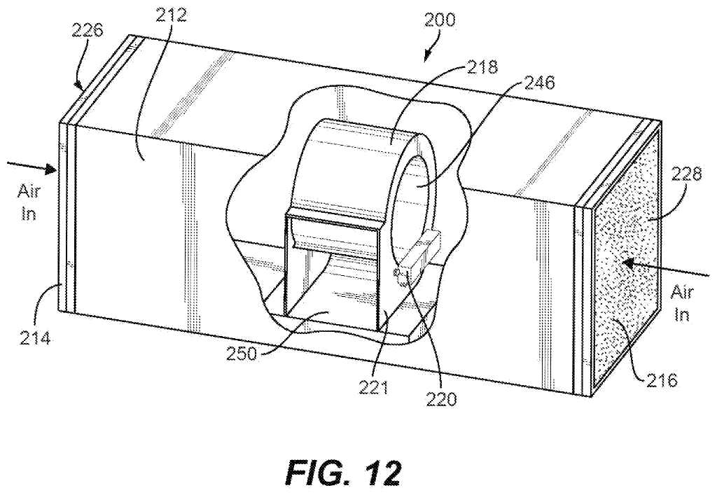

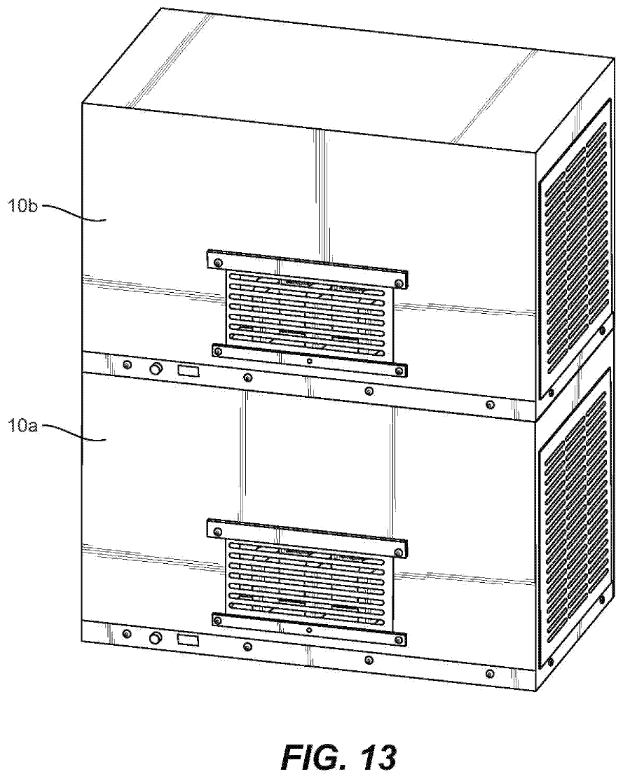

Referring to FIG. 12, an alternate embodiment of an air purification device 200 is similar to air purification device 10, except that no silencer duct is provided, ionizer 220 is mounted to one side of blower 218 (e.g., at or adjacent both intake portion 246 and exhaust portion 250 of blower), and housing 212 defines a larger interior space between filters 214, 216 at inlets 226, 228. It will be appreciated that in this embodiment, intake air which is filtered is ionized within housing 212 as it becomes exhaust air (e.g., after it enters the interior of housing 212 as filtered air). The ionized filtered exhaust air exits exhaust portion 250 of blower 218 and is immediately ejected through an outlet in the housing (not shown). Referring to FIG. 13, in another embodiment, air purification devices 10a, 10b are similar or identical to air purification device 10, and mounted or stacked on top of one another. This embodiment may be utilized where there is a particularly high concentration of contaminated air and even more rapid purification is desired.

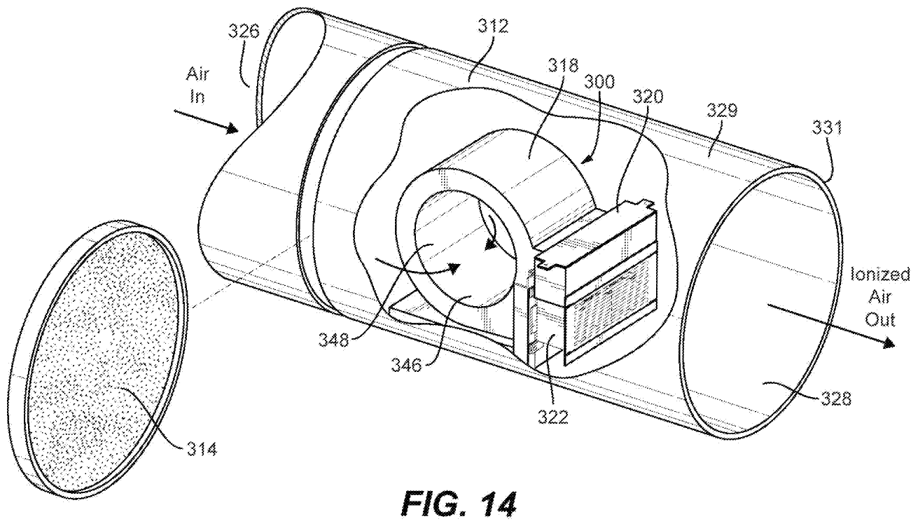

While air purification device 10 has been described herein as a standalone unit separate and apart from, for example, an HVAC system, it will be appreciated that the air purification devices and systems described herein may alternatively or additionally be utilized within a fluid duct of a ventilation system. By way of example, as shown in FIG. 14, in another alternative embodiment, purification device 300, silencer duct 322, and ionizer 320 may be arranged in axial alignment with a cylindrical pipe 329, and mounted within a cylindrical housing 312 coaxial with cylindrical pipe 329.

In this embodiment, a filter 314 is installed adjacent at an air inlet section 326 on one side of cylindrical housing 312 upstream of blower 318, and opposite end 331 of cylindrical housing 312 is open to discharge ionized filtered air in the manner described above. In this embodiment, air at inlet 326 is filtered by filter 314 and then sucked around the side of blower 318 to cylindrical channel 348 defined by intake portion 346 of blower 318. The filtered air sucked in at intake portion 346 is then forced through silencer duct 322 as described above, and discharged from pipe 329 as shown. In such embodiments, other blowers may be utilized where cylindrical channel 348 of intake portion 346 is coaxially aligned with pipe 329. It will be appreciated that in such embodiments, air purification device 300 could be placed at one or more vents of an HVAC system so that exiting air is further purified, filtered, ionized, and immediately ejected into an occupied space.

While various features have been disclosed in different embodiments herein, it will be appreciated that any combination of any number of features in the embodiments described herein may be combined, and that the invention is not limited to the specific combinations of features disclosed in the drawings or description thereof. The invention has been described in the context of a number of embodiments and multiple variations and examples thereof. It is to be understood, however, that other variations, shapes, materials, methods of manufacture, features, and structures may be employed without departing from the spirit of the invention.

Therefore, it is intended that the appended claims as presented or subsequently amended be interpreted as including the embodiments described herein, the alternatives mentioned above, and all equivalents thereto.

What is claimed is:

1. An air purification device for purifying and recirculating air, comprising:
a housing having an inlet configured to guide room air into the housing, and an outlet configured to guide ionized air out of the housing;
a filter mounted within the housing adjacent the inlet;
a blower mounted within the housing and defining an intake portion and an exhaust portion, wherein the blower is configured to draw the room air through the inlet and the filter to form filtered air within the housing which enters the intake portion, and to push the filtered air through the exhaust portion toward the outlet of the housing; and
an ionizer mounted within the housing and configured to ionize particles within the filtered air in the housing to generate the ionized air guided out of the housing.

2. The air purification device of claim 1, wherein the ionizer is mounted at or adjacent the blower.

3. The air purification device of claim 2, wherein the ionizer is mounted at or adjacent the exhaust portion of the blower.

4. The air purification device of claim 3, further comprising:
a silencer duct mounted within the housing in fluid communication with the exhaust portion of the blower, wherein the ionizer is mounted to the silencer duct, and the silencer duct is configured to direct the filtered air pushed through the exhaust portion of the blower, through a portion of the ionizer, to the outlet of the housing.

5. The air purification device of claim 4, wherein the silencer duct includes at least one baffle configured to slow filtered air exiting the exhaust portion of the blower such that a noise level of airflow within the air purification device is reduced.

6. The air purification device of claim 5, wherein the silencer duct includes a support surface for mounting the ionizer, the ionizer includes at least one brush for generating ions in the filtered air exiting the exhaust portion of the blower, and the support surface defines a cutout for receiving the at least one brush such that the at least one brush extends downwardly through the cutout.

7. The air purification device of claim 6, further comprising:
a nozzle mounted within the silencer duct and configured to spray water into the filtered air being ionized by the ionizer in the silencer duct.

8. The air purification device of claim 7, wherein the at least one baffle includes a pair of baffles, and the at least one brush includes a first brush operatively disposed above and horizontally between the pair of baffles.

9. The air purification device of claim 7, wherein the at least one baffle includes a middle baffle and two end baffles on opposite sides of the middle baffle, the at least one brush includes a first brush and a second brush, the first brush is operatively disposed above and horizontally between the middle baffle and one of the two end baffles, and the second brush is operatively disposed above and horizontally between the middle baffle and the other of the two end baffles.

10. The air purification device of claim 1, further comprising:
a plurality of wheels supporting the housing and configured to mobilize the air purification device along a floor.

11. The air purification device of claim 10, further comprising:
a controller operatively associated with at least one of the plurality of wheels; and
a remote computing device operatively associated with the controller, wherein the remote computing device is operable by a user to mobilize the air purification device along the floor.

12. The air purification device of claim 1, further comprising:
a mounting structure for mounting the device to a wall or ceiling.

13. The air purification device of claim 1, wherein the ionizer operates at a voltage less than or equal to 11.7 electron volts, and the ionizer produces ionized air in the housing having an ozone level in a range of 0 to 0.05 ppm.

14. An air purification system for purifying and recirculating air, comprising:
a first array of air purification devices according to claim 1, wherein a first of the first array of purification devices and a second of the first array of purification devices are arranged such that a portion of ionized air exiting the outlet of the housing of the first of the first array of purification devices is received in the inlet of the second of the first array of purification devices.

15. An air purification system according to claim 14, further comprising:
a second array of air purification devices according to claim 1, wherein a first of the second array of purification devices and a second of the second array of purification devices are arranged such that a portion of ionized air exiting the outlet of the housing of the first of the second array of purification devices is received in the inlet of the second of the second array of purification devices,
wherein the first and second array of air purification devices are standalone units spaced apart from one another, and
wherein one of the first array of air purification devices and one of the second array of air purification devices are fluidly coupled by a fluid duct.

16. A method of purifying and recirculating air, comprising:
drawing room air through an inlet of a housing and a filter mounted in the housing to form filtered air within the housing;
drawing the filtered air to an intake portion of a blower in the housing;
pushing, by the blower, the filtered air toward an outlet of the housing;
ionizing, via an ionizer, the filtered air in the housing to form ionized air; and
guiding, via an outlet in the housing, the ionized air out of the housing,
wherein the outlet is at or adjacent the blower.

17. The method of claim 16, wherein the ionizer is configured to ionize the filtered air with ozone in a range of no more than 0.005 parts per million.

18. The method of claim 16, wherein the blower is configured to push the ionized air out of the housing such that the flow rate of electrically induced ions pushed out of the housing is in a range of 340,000,000 to 400,000,000 ions/cm³/second.

19. The method of claim 16, further comprising:
guiding the filtered air exiting an exhaust portion of the blower through a silencer duct, wherein the ionizer is mounted to the silencer duct.

20. The method of claim 19, wherein the silencer duct includes a support surface for mounting the ionizer, the ionizer includes at least one brush for generating ions in the filtered air exiting the exhaust portion of the blower, and the support surface defines a cutout for receiving the at least one brush such that the at least one brush extends downwardly through the cutout.
